(19) United States
(12) Patent Application Publication
(10) Pub. No.: US 2003/0213052 A1
(43) Pub. Date: Nov. 20, 2003

(54) Diving mask

(75) Inventor: Pan Chen-Lieh, Yuan Shan Shiang (TW)

Correspondence Address: BRUCE H. TROXELL, SUITE 1404, 5205 LEESBURG PIKE, FALLS CHURCH, VA, 22041, US

(73) Assignee: QDS Injection Molding LLC.

(21) Appl. No.: 10/147,892

(22) Filed: May 20, 2002

(51) Int. Cl. A61F 9/02

(52) U.S. Cl. 2/428

(57) ABSTRACT

A diving mask which comprising: A framing apparatus, provided in both sides of nose portion, at least giving front glasses mounted in front frame; A mask apparatus which made by soft and flexible material, having a nose portion and edging piece extending along the facial shape, on the both side of nose portion under the front frame, integrally forming lower frames and each frame providing a lower glasses.

### FIELD OF THE INVENTION

The present invention is relating to a diving mask, especially a diving mark with the lower tens, which enable the diver to look downwardly and expand the view to diver's pectoral and waist regions.

### BACKGROUND OF THE INVENTION

In diving activity, especially in scuba diving, unavoidably use the diving mask. Due to the diving activity usually under the self-protected situation, therefore, personal security devices are extremely important. Among these devices, the diving mask is the most important and necessary one.

The conventional diving mask, such as U.S. Pat. No. 6,341,863, disclosing a diving mask having the front lens and side lens which providing wider view for diver. Beside this, there are series of diving mask in market which having the front lens and side lens which shown as FIG. 3.

Actually, when diving, excepting the diver viewing the circumstance around him through front lens and side lens, he must possess his devices at any time. For example, looks downwardly to observe weight belt, buoyancy control harness, tangles hoses and relating devices adopted in waist. Under this circumstance, looking downwardly is unavoidable.

Therefore, U.S. Pat. No. 5,345,615 disclosed a diving mask having the lens for looking downwardly. In this patent, by the very complicate structure, under the front lens, provided a pair of second lens which expanding the view to look down the diver's pectoral region and further forming a six window mask. That means, this kind of mask having a pair of front lens, a pair of side lens, and a pair of lower lens (or auxiliary lens) which under the front lens. Although it can achieve the basic function of looking downwardly, but the disadvantage is that the frame of lower lens were made by the material different from the mask and must be combined by glue or sealed high frequency wave, which make the production procedure more complete and higher cost.

### SUMMARY OF THE INVENTION

In order to overcome the disadvantage, based on the long-termed experience in research and development as well as producing of diving mask, and through the intensive experiment, then matured the present invention of diving mask. The present invention provides a diving mask having six views and the frames of lens are formed integrally.

### BRIEF DESCRIPTION OF THE DRAWINGS

FIG. 1 is the perspective and explored view of the present invention.

FIG. 2 is the perspective view of the present invention.

FIG. 3 is the drawing of one of the conventional diving mask.

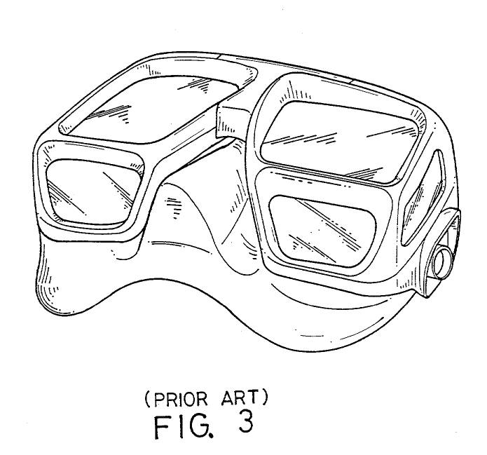

### DESCRIPTION OF THE PREFERRED EMBODIMENT

Referring to the drawings, basically, the diving mask of the present invention comprising a framing apparatus 1 and a masking apparatus 2.

Said framing apparatus 1 composed by frames 11 and lens or glasses 12 and which combining with masking apparatus during practice. Said frames 11 has at least two front frames with glasses (or lens) 12. As shown in drawings, the frames 11 of diving mask of the present invention further comprising a pair of side frame which extending from the front frames. Said glasses 12 are filled to each frame closely and prefer but not limited made by transparent material such as reinforced glasses. Conventionally, the framing apparatus is adopted in the both sides of nose portion 21 of said masking apparatus 2.

Masking apparatus 2 covering diver face closely when wearing, therefore, its shape is corresponding to the nose and eyes portion of face. In the middle, there is a swell up nose portion 21 which enable to locate the nose of diver. Corresponding to diver's face, integrally extends the edging piece 22 toward bottom, side as well as back directions.

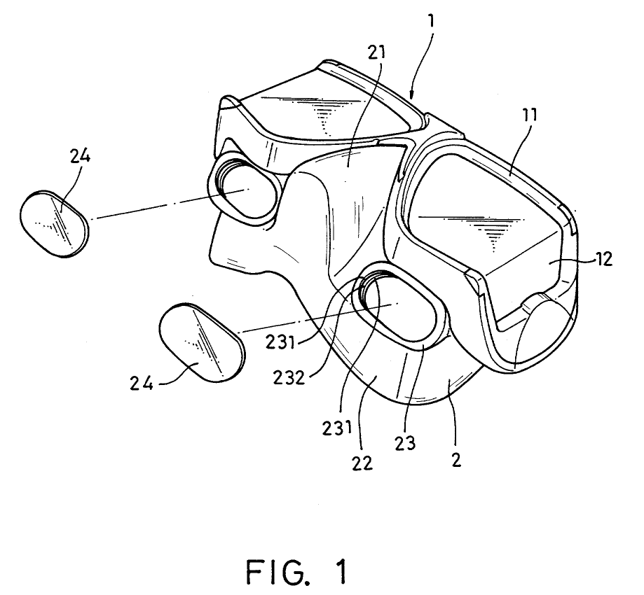

The significant difference between the present invention and conventional diving mask is the frames 11. As showing in FIG. 1 inwardly extending from said lower edging piece 22 of front frame, forming two frame rim 231 and further forming a frame groove 232 for lower lens mounting. As mentioned before, due to the mask apparatus 2 is made by soft and flexible material, therefore, the size of groove 232 is corresponding to the size of lower glasses 24, whereby the lower lens 24 can be fixed and positioned therein. For better protective viewing effect, the material of lower glasses 24 is but not limited to reinforced glass.

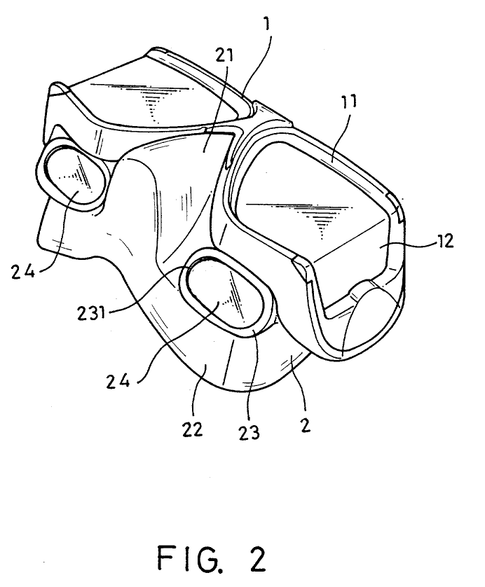

Referring to FIG. 2, lower glasses 24 can be mounted and fixed into the frame groove 232 and become a complete diving mask.

In practice, the lower glasses 24 can be placed within the pre-design mold and forming integrally with mask apparatus 2. By this way, the lower glasses can be integrally surrounded by the ended by mask apparatus 2 and combined in more closer.

Therefore, by the practice of the present invention, the diving mask with lower glasses can be easily obtained and it is not easily achieved by the convention diving mark. In other words, the present invention is a breakthrough of diving mask technology.

The foregoing description of a preferred embodiment of the present invention has been by way of example and not of limitation. Understandably, many changes and modifications may be made thereunto within the scope of the inventive concept. It is understood that the invention scope should be that defined by the appended claims.

What is claimed is:

1. A diving mask which comprising:
A framing apparatus, provided in both sides of nose portion, at least giving front glasses mounted in front frame;
A mask apparatus which made by soft and flexible material, having a nose portion and edging piece extending along the facial shape, on the both side of nose portion under the front frame, integrally forming lower frames and each frame providing a lower glasses.

2. A diving mask as claimed in claim 1, wherein said frame apparatus further provides side frames with side glasses which extending backward from the front frames.

3. A diving mask as claimed in claim 1, wherein said lower glasses are formed integrally with said mask apparatus.

4. A diving mask as claimed in claim 1, wherein said lower frames are composed by two frame rims and thereby forming a frame groove which enable the lower glasses mounted therein.

5. A diving mask as claimed in claim 1, wherein said lower glasses placed in the mold of said lower frames and forming integrally.

6. A diving mask as claimed in claim 1, wherein said lower glasses are made by transparent and rigid material.

7. A diving mask as claimed in claim 1, wherein said lower glasses are made by reinforced glasses.
